(19) United States
(12) Patent Application Publication
(10) Pub. No.: US 2020/0281877 A1
(43) Pub. Date: Sep. 10, 2020

(54) METHOD FOR PREVENTION AND TREATMENT OF ACNE, ECZEMA, PSORIASIS, AND RELATED DERMATOLOGICAL CONDITIONS WITH COMPOSITION COMPRISING A SPHINGOLIPID AND SULPHUR COMPOUNDS

(71) Applicants: José L. CALDERÓN, Howard Beach, NY (US); Clark L. SWANSON, Henderson, NV (US)

(72) Inventors: José L. CALDERÓN, Howard Beach, NY (US); Clark L. SWANSON, Henderson, NV (US)

(21) Appl. No.: 16/682,593

(22) Filed: Nov. 13, 2019

Related U.S. Application Data
(60) Provisional application No. 62/768,307, filed on Nov. 16, 2018.

(51) Int. Cl. A61K 31/164; A61K 31/10; A61K 33/30

(52) U.S. Cl. CPC A61K 31/164; A61K 33/30; A61K 31/10

(57) ABSTRACT

A composition for use in treating skin disorders and especially acne without the side effects of steroids, antibiotics, anti-fungal, and other treatments (e.g. UV radiation, light therapy, etc.). The orally-administered composition is a compound of sphingolipid and an ingestible sulfur (including any sulfur compound, sulfur-containing moiety, or sulfur salt). In a preferred embodiment the present composition also includes an adjuvant, preferably zinc and/or a proanthocyanidin, preferably from grape seed extract, and other excipients for taste, consistency and/or coloring. The above-described composition is orally administered in tablet form pursuant to a prescribed protocol of therapeutic doses to a subject.

### CROSS-REFERENCE TO RELATED APPLICATION(S)

The present application derives priority from U.S. provisional application Ser. No. 62/768,307 filed 16 Nov. 2018.

### BACKGROUND OF THE INVENTION

### 1. Field of the Invention

The invention generally relates to the treatment of skin conditions in subjects having, or at risk for developing skin conditions, such as acne, eczema and psoriasis, and other related dermatoses. In particular, the invention provides methods for treating skin, which is either damaged or undamaged, using compositions containing a compound of a sphingolipid and sulfur that are orally administered.

### 2. Description of the Background

Skin disease is highly prevalent in the United States (U.S.) typically affecting about 60% of persons. The prevalence of skin disease requiring medical intervention is about 20% and consequently about 10% of a primary care physician's workload and 6% of hospital outpatient referrals are attributable to them. Among all skin disease cases about 60 million are attributable to acne that may present at various stages of severity, from mild or occasional break-outs to the severe inflammation and infection in gram negative folliculitis. After acne, atopic eczema (atopic dermatitis) and psoriasis are most prevalent. The following summarizes each of these skin diseases.

Acne

Acne is the most common skin condition in the United States affecting up to 60 million Americans annually. It is highly prevalent in childhood, adolescence, and young adulthood typically affecting 60 percent of the population at any given time including more than 85% of teenagers and 10% of adults. Although the highest prevalence is typically seen in adolescence, remission of this dermatosis may be followed by outbreaks later in life and precipitated by conditions associated with hormonal changes, such as pregnancy, or those associated with hormonal dysregulation, such as in Cushing's Syndrome and obesity, as well as environmental and psychological stress.

Acne is a folliculitis characterized by inflammation of hair follicles and underlying infection, such as bacterial or fungal, that most often arises in puberty when the body's production of hormones, such as dihydrotestosterone or testosterone, increase rapidly. Specifically, this condition is characterized by the presence of pro-inflammatory entities that alter the integrity of the dermal hair follicular walls lying within the skin's epidermal layer and secondary infections typically caused by the anaerobic, gram positive bacteria Propionibacterium acnes (P. acnes) but is also caused by the Malassezia fungus, which is strongly associated with seborrheic dermatitis.

P. acnes is normally found on the skin's surface and although it is a gram-negative, anaerobic bacteria it tolerates aerobic environments well, such as skin surfaces. Due to hormonal elevation such, such as occurs in puberty, sebaceous glands increase lipid (sebum) production and greater amounts of lipid are excreted into hair follicles making the skin oilier. Moreover, P. acnes bacteria migrate well though lipids greater number of bacterial colonies grow within the hair follicle.

Complicating the increasing number of P. Acnes bacterial colonies in hair follicles is the sebaceous plugging of hair follicles at their skin surface openings creating a less oxygenated and more anaerobic environment that is favorable for P. acnes growth. This foments the growth of a nidus of infection within obstructed follicles that promotes oxidative stress, immune dysregulation that triggers the release of reactive chemical species, such as ROS and NR-kB, that trigger inflammation responses and may further promote oxidative stress.

These aforementioned conditions are commonly treated with topical and oral antibiotics. Unfortunately, treatment with antibiotics has side effects and is generally not recommended for long term use due to the microbiome disruption within the intestines. Despite the ill effects of long-term use, dermatological treatment has relied on antibiotics in the treatment of acne with drugs such as doxycycline HCL that is administered in a prophylaxis dose of 40 mg per day.

Other pharmaceutical options include isotretinoins, vitamin A based retinoids, that are used for the treatment and prevention of severe acne but has harsh side effects, such as complications of the liver and other organs. Oral isotretinoin suppresses sebaceous gland activity and so indirectly reduces inflammatory lesions; nevertheless, isotretinoin is not a curative drug and discontinuation of the treatment is frequently followed by recurrence in the absence of appropriate maintenance treatment. Besides, due to potentially serious side effects (birth defects, vision problems, mental health problems, hair loss, vision problems, etc.), isotretinoin is not routinely prescribed to patients. Therefore, acne patients would welcome an alternative, safer, and long-term effective treatment.

Eczema (Atopic Dermatitis)

Eczema (atopic dermatitis) is characterized by three conditions; genetic predisposition, family history of allergic conditions, such as asthma or allergic rhinitis, and loss of the epidermal skin barrier. Clinically it is characterized by exacerbation and remission of patches of dry, scaly, rough, discolored and inflamed areas of skin. Eczema is also known to be exacerbated by environmental factors, such as irritants and pollution, as well as by emotional and psychological triggers.

A flare up can go into remission only to recur at any time, usually around the flexor areas of the arms and legs and the head of children that is commonly called ‘cradle cap’. Chronically, eczema flares are associated with small raised exudative lesions that are fluid filled and partially drain when scratched increasing the risk of infection. To treat this topical antibiotics and anti-inflammatory steroids, such as hydrocortisone, are most often prescribed.

Antibiotics have been used in the treatment of eczema for decades. This treatment is ex-post and meant to treat the symptomatic sequalae of eczema but do not address its root cause, such as genetic predisposition. To treat severe outbreaks of eczema oral steroids may be used but carry the risk of side effects, such as immune suppression, elevated blood sugar and blood pressure. The mechanism of action includes repression of abnormally functioning T cells and macrophages, cytokines, such as NL-kB, IL-4, IL-5, immunoglobin IgE, and B cells and eosinophils that are types of white blood cells directly involved in allergic reactions.

These chemical moieties and cells are over produced by dysregulation of the body's immune system. (Ohmen J D et al., J Immunol., 154: 1956-1963, 1995-Overproduction of IL-10; Hamid Q et al., J Clin. Invest., 94: 870-876, 1994-Cytokine expression; which are incorporated by reference herein in their entireties). Steroid therapy is not recommended for children who suffer from such skin conditions as side effects, such as immunosuppression, inhibited growth, and adrenal fatigue have been observed.

Psoriasis

Psoriasis is a chronic autoimmune skin disease characterized by patches of abnormal skin that red, dry and itchy and scaly. These psoriatic skin changes may be triggered at areas of skin subjected to environmental irritant factors. Psoriasis is generally believed to have a genetic basis. The observation that an identical twin has a three-Told risk of developing psoriasis should the other twin have the condition as opposed to a significantly lesser risk for a non-identical twin gives credence to this condition having a genetic basis.

There are five main types of psoriasis: plaque, guttate, inverse, pustular, and erythrodermic. Plaque psoriasis, also called psoriasis vulgaris, accounts for about 90% of cases and typically presents as red patches over which white scales form. Symptoms often worsen during winter months and with consumption of certain medications, such as beta blockers that are commonly used for treating high blood pressure, and non-steroidal anti-inflammatory drugs (NSAIDS), such as ibuprofen and naproxen that are used for pain relief. Skin infections and psychological stress have been implicated as triggers for the exacerbation of psoriasis.

The pathological underpinning of psoriasis is a person's skin cells being targeted and harmed by their own immune system. Diagnostic differentiation of psoriasis types is typically based on clinical evaluation of a patient's signs and symptoms. The Mayo Clinic describes this condition as one in which “signs and symptoms are different for everyone”. [https://www.mavoclinic.org/diseases-conditions/psoriasis/symptoms-causes/syc-20355840] Common signs and symptoms include red patches of skin covered with thick, silvery scales, small scaling spots (commonly seen in children), dry, cracked skin that may bleed, itching, burning or soreness, thickened, pitted or ridged nails and may be associated with swollen and stiff joints (psoriatic arthritis). Most types of psoriasis go through cycles, flaring for a few weeks or months, then subsiding for a time or even going into complete remission.

What is needed is a method to improve the human condition, while at the same time not disrupting the body's own microbiome or having other unwanted side effects. The current method contributes to alleviating skin disease conditions that would also improve the overall quality-of-life of the recipient. The present composition acts by facilitating corrections to the loss of balance in the oxidative environment, by the reduction of free radical species, such as ROS, the inhibition of cytokine release, and the repair of the skin barrier by restoring the skin's epidermal cellular architecture.

This mechanism of action is relevant to all inflammatory dermatosis related skin conditions, such as acne, eczema, and psoriasis. Moreover, the current invention would indirectly help reduce the emotional and psychological sequalae of skin disease (i.e., depression, anxiety, social isolation, lower self-esteem, etc.), that are themselves triggers for skin disease, thereby reducing symptoms and suffering while also improving recovery time. The aforementioned conditions of the skin tissues are improved by the improvement of moisture retention and toxic flushing of the organ by the hydration and rehydration process that results in improved skin barrier performance. [Clinical Dermatology vol. 31 (2013) 677-700].

The pathogenesis and progression of skin disease is underpinned by oxidative stress and involves the introduction of free radical components such as reactive oxygen species (ROS), reactive nitrogen species (RNS) or lipid peroxidation (LPO). Antioxidant-related therapies such as the use of nutraceuticals are of particular growing interest in the segment of the global population with skin conditions. While the topical application of medicine and other therapeutic compounds have been attractive for the administration of drugs, the ability to deeply penetrate the skin's multiple layers has underperformed and few have provided proven scientific or conclusive results.

The epidermis is the most superficial layer of skin and the stratum corneum, it outer most layer, provides a vital permeability barrier against water loss, environmental xenobiotics and harmful microbes. The epidermis is comprised of a lipid rich lamellar matrix that embeds corneocytes generated in its lower layers. These corneocytes are enucleated as they migrate from the more acidic lower epidermal environment to the more alkaline upper epidermal environment during which time they accumulate ceramides due to increased ceramide synthesis.

Sphingolipids are a class of lipids containing a backbone of sphingoid bases, a set of aliphatic amino alcohols that includes sphingosine. They play important roles in signal transduction and cell recognition. Simple sphingolipids include the sphingoid bases and ceramides

Ceramides are a family of waxy lipid molecules comprised of sphingosine and fatty acids are crucial for structuring and maintaining skin barrier integrity. They are the major component of the epidermal lamellar matrix that are highly ordered intercellular lamellar structures. Squamous cells of the stratum corneum are comprised of 50% ceramides along with 25% cholesterol, and 15% free fatty acid. [The Clinical Relevance of Maintaining the Functional Integrity of the Stratum Corneum in both Healthy and Disease-affected Skin, James Q. Del Rosso, D O, FAOCD and Jacqueline Levin, et. al., J Clin Aesthet Dermatol. 2011 Sep.; 4(9): 22-42. (https://www.ncbi.nlm.nih.gov/pmc/articles/PMC3175800/). Ultra-long chain (C28-C36) ceramides are key components of extracellular lipid lamellae of the stratum corneum. Together with cholesterol and saturated fatty acids, ceramides create water-impermeable skin, a protective organ that prevents excessive exit of water due to evaporation as well as an entry barrier against microorganisms.

Sphingolipid ceramide N-deacylase (SCDase) is an enzyme that catalyzes the synthesis of glycosphingolipids (GSLs). It plays a crucial role in maintaining GSL homeostasis since it catalyzes the reversible hydrolysis/synthesis of the amide linkage between the fatty acid and the sphingosine base in the ceramide moiety of GSLs. In the case of GSLs exohydrolases, acting at acidic pH optima of the lower layers of the epidermis, cause the step wise release of monosaccharide units from the end of the oligosaccharide chains, leaving just the sphingosine portion of the molecule, which may then contribute to the generation of ceramides.

Constitutive degradation of sphingolipids and glycosphingolipids takes place in the acidic subcellular compartments of the epidermis. SCDase catalyzes reversible reactions iii which the amide linkage in glycosphingolipids is hydrolyzed or synthesized. The optimal pH values for the hydrolytic and synthetic activity of SA_SCD were pH 6.0 and pH 7.5, respectively. Both activities were strongly inhibited by bivalent zinc (Zn²⁺).

Ceramide and cholesterol decline in the stratum corneum of humans due to aging. In eczema the stratum corneum is hypoplastic and/or disintegrated and in psoriasis the stratum corneum is hyperplastic and exudative. Both dermatoses are characterized by compromised water exit permeability and microbial ingress barrier.

Sulfur has been used in skincare in ancient Indian, Egyptian, Greek and Chinese civilizations. Although it has fallen out of favor in shampoo form, sulfur is still widely used to treat a number of skin conditions in cream, lotion and soaps. The principal dietary sources of sulfur, the amino acids methionine and cysteine, may not always be consumed in adequate amounts to meet sulfur requirements.

Sulfur has a long history of use in skin conditions, and any effect is likely due to the anti-infective properties making it potentially useful in seborrheic dermatitis and acne. Sulfur is an excellent anti-bacterial and antifungal agent and so is most effective in skin conditions associated with underlying infections, such as acne, eczema and psoriasis. It is converted to hydrogen sulfide by bacteria for energy production that quickly becomes toxic to them. Hydrogen sulfide kills a range of organisms, including bacteria, fungi, yeasts, and parasites making it an effective preservative and anti-infective.

Anecdotal evidence online indicates sulfur is exceptionally effective at treating acne. Sulfur soaps, face masks, creams and shampoos can be widely found in alternative health stores, pharmacies, and online retailers in concentrations up to 10%. In acne, sulfur helps dry the skin, slowing sebum production and its anti-infective properties are effective against P. acnes bacteria. Sulfur compounds also inhibit the growth of Malassezia fungus, which is strongly associated with seborrheic dermatitis.

A 2012 report in the Journal of Medicinal Chemistry determined that sulfonamides were as effective in preventing the fungus' growth than the pharmaceutical ketoconazole 2% prescription strength—the active ingredient found in Nizoral. Sulfur is also mildly keratolytic and can be used as a treatment for psoriasis.

The naturally occurring organosulfur compound, methylsulfonylmethane (MSM), is available as a dietary supplement and has been associated with multiple health benefits. Many medicines contain sulfur preservatives to prevent bacterial growth, improving shelf-lives. These are listed on the ingredient label, with common examples including sulfur dioxide, sodium sulfite, sodium metabisulfite. Each of these has been categorized as ‘generally recognized as safe’ by the US Food and Drug Administration (FDA), and are widely used in tablets and injectables. Zinc is an essential mineral required during prenatal and postnatal development. Zinc deficiency affects about two billion people in the developing world and is associated with many diseases. In children, deficiency causes growth retardation, delayed sexual maturation, infection susceptibility, and diarrhea. Zinc, being an important mineral, plays a vital role in protein synthesis and helps regulate the cell production in the immune system of the human body by zinc signaling to communicate with other cells.

Zinc acts as an antioxidant and is involved in some of the biochemically decisive reactions in the body, including protein synthesis, enzymatic function, and carbohydrate metabolism. It is mostly found in the strongest muscles of the body and is found in especially high concentrations in the skin.

Zinc helps to treat acne by enzymatic functions regulating and controlling the amount of testosterone in the body, thereby regulating sebum production which plays a dominant role in causing acne. Furthermore, the concentration of zinc in serum and hairs has been demonstrated to be significantly lower in children aged 2 to 12 years with bronchial asthma and atopic dermatitis compared to normal skin.

Zinc is also an inhibitor of NADPH oxidase, which is involved in generation of free radicals that contribute to development of oxidative stress. Therefore, zinc is generally considered to be an antioxidant. In most single-tablet, over-the-counter, daily vitamin and mineral supplements, zinc is included in such forms as zinc oxide, zinc acetate, or zinc gluconate.

Zinc participates in the regulation of cell proliferation by being essential to enzyme systems that influence cell division and proliferation. Enzymes with a zinc atom in the reactive center are widespread in biochemistry, such as sphingolipid ceramide N-deacylase (SCDase), involved in maintaining a homeostasis in the synthesis of glycosphingolipids (GSLs). Sphingolipid ceramide N-deacylase (SCDase) is an enzyme that can serve as a biocatalyst in the enzymatic synthesis of GSLs since it catalyzes the reversible hydrolysis/synthesis of the amide linkage between the fatty acid and the sphingosine base in the ceramide moiety of GSLs.

Moreover, in-vitro studies have shown that zinc inhibits hydroxylation of ceramide in more acidic pH environments, such as is found in the lower epidermal layer, and synthesis of ceramide in more alkaline pH environments, such as is found in the upper epidermal layers. This is consistent with the role of zinc in the hydrolysis/synthesis of the amide linkage between the fatty acid and the sphingosine base in the ceramide moiety of GSLs.

Proanthocyanidins for oral administration have been derived from certain herbs such as burdock root, French tree bark, resveratrol, and grape seed extracts. Grape seed extract, for example, contains oligomeric proanthocyanidins or OPC's which are found to possess and estimated 20 times the antioxidant power of Vitamin C and 50 times that of Vitamin E. Some studies have suggested that the synergistic use of antioxidants provide an important contribution to the treatment of skin disease. For example, a trade name of French tree bark branded Pycnogenol® has been used in a variety of nutraceuticals.

The OPC's of Pycnogenol® is 70%, yielding good proanthocyanidin coverage to reduce oxidative stress. This is found in a proprietary blend from Source Naturals™ called Pycnogenol Complex™ that provides vitamins, minerals and herb extracts to help protect the body against free radicals. The complex contains Pycnogenol®, proanthodyn, quercetin, Ginkgo Biloba extract, Green Tea extract, Bilberry extract, Silymarin, Tumeric extract, Hawthorn Berry extract, Rosemary extract, vitamin C (in the form of zinc and magnesium ascorbates), and magnesium.

Through the iterative references that are disclosed in the literature, in addition to various supplements and nutraceuticals marketed to consumers today, none of these disclose or suggest the combination use of a sphingolipid with sulfur as a dermatological agent to be used for managing specific dermatological conditions. What is needed is a method and composition to improve the human skin condition, while at the same time not disrupting the body's own microbiome or having other unwanted side effects.

Indeed, such an approach would not alleviating skin disease but improve the overall life of the recipient. The present invention provides such a composition that acts by facilitating corrections to the physical condition of the skin that has been impacted by the disease. The result is a reduction in ROS, inhibiting cytokine release and thereby reducing suffering of the patient while also improving recovery time. Equally important is the longevity of sustained recovery with use. The condition of the skin tissues results in improved barrier performance by the addition of moisture retention and toxic flushing of the organ by the hydration and rehydration process.

### SUMMARY OF THE INVENTION

It is, therefore, an object of the present invention to provide a composition for use in dermatological application which is effective for treating skin disorders without the side effects of steroids, antibiotics, anti-fungal, and other treatments (e.g. UV radiation, light therapy, etc.). The need for treating these skin conditions also relates to the delivery of medicine at the point of insult—topical applications of creams, lotions, steroids, or other modalities have not proven effective long term.

According to the present invention, the above-described and other objects are accomplished by providing an orally-administered composition comprising a compound of sphingolipid plus an ingestible sulfur, sulfur compound, sulfur-containing moiety, or sulfur salt, for the prevention and treatment of acne, eczema, psoriasis and other skin conditions. In a preferred embodiment the present composition also includes an adjuvant and other excipients for taste, consistency and/or coloring.

The sphingolipid is preferably a phytosphingosine such as ceramide (2-amino-4-octadecene-1,3-diol), and most preferably rice-derived phytosphingosine, although a variety of other choices for phytoceramide can be made, such as those derived from potatoes or wheat.

The sulfur, sulfur compound, sulfur moiety or sulfur salt, may be any ingestible sulfur source, preferably Methylsulfonylmethane (MSM), although a variety of other choices can be made.

The composition may be comprised of one or more adjuvants and one or more excipients. The adjuvant in the preferred embodiment is zinc. Zinc is an immunogenicity reactive compound utilized to draw a cellular response from the protein. In this case, “scientists have determined in human cell cultures and animal studies that a protein lures zinc into key cells that are first responders against infection.” Zinc helps against infection by tapping brakes in immune response. This process is a vital biological function to fight infection and balance immune response. [News Release, “Zinc helps against infection by tapping brakes in immune response”, Ohio State University (Feb. 7, 2013).]

In another aspect, provided herein is a method comprising orally administering the above-described composition pursuant to a prescribed protocol of therapeutic doses to a subject at risk for having or having skin diseases such as acne, eczema, psoriasis or other related dermatoses.

### DETAILED DESCRIPTION OF PREFERRED EMBODIMENTS

Generally, the invention is a composition for use in dermatological application which is effective for treating skin disorders without the side effects of steroids, antibiotics, anti-fungal, and other treatments (e.g. UV radiation, light therapy, etc.). The invention also comprises a method for treating these skin conditions by periodic oral ingestion. The composition actives include a sphingolipid together with a suitable ingestible sulfur, sulfur-containing compound, organosulfur compound, sulfur salt or other organosulfur moiety. The sphingolipid is preferably a ceramide. Ceramides are a family of waxy lipid molecules composed of sphingosine and a fatty acid. Ceramides are found in high concentrations within the cell membrane of eukaryotic cells, since they are component lipids that make up sphingomyelin, one of the major lipids in the lipid bilayer. Research has shown that topical application of ceramides to the skin surface can improve barrier function, but the present invention relies on ingested ceramide delivery pathways for improved effect.

The ceramide most preferably comprises phytosphingosine (2-amino-4-octadecene-1,3-diol), and most preferably rice-derived phytosphingosine, although a variety of other choices for phytoceramide can be made, such as those derived from potatoes or wheat for example.

The sulfur may comprise any ingestible organosulfur compound, sulfur salt or other organosulfur moiety. Organosulfur compounds are organic compounds that contain sulfur such as cadmium sulfide or zinc pyridinium-1-thiol N-oxide (Zpt), triclosan, halocarban, menthone, undecylene acid, resorcinol, isopropylmethylphenol and salicylic acid. Organosulfur moieties include sulfur-containing proteinogenic amino acids such as cysteine or methionine.

A zinc adjuvant is also used in the preferred embodiment.

The composition may optionally include one or more excipients for oral administration. A “physiologically acceptable” excipient is any inert constituent that improves suitability for oral administration such as water, buffered solutions and glucose solutions, among others, stabilizers, preservatives, diluents, emulsifiers or lubricants. In particular, suitable excipients include, but are not limited to, Tween 20, sucrose, L-histidine, polysorbate 20 and serum.

In another aspect, provided herein is a method comprising orally administering the above-described composition pursuant to a prescribed protocol of therapeutic doses to a subject having or at risk of skin condition (e.g. acne, psoriasis, or eczema).

The above-described therapeutic composition in specifically comprises quantities of constituents in conformance with the following Table:

| | | Preferred | | Acceptable |
|---|---|---|---|---|
| Constituent | Name | Amount | | Range |
| Sphingolipid | phytoceramide such as | 40 | mg | +/−50% |
| | Ceramide-Rx ® or | | | |
| | Ceramide-PCD | | | |
| Sulfur | Pure Methylsulfonyl- | 300 | mg | +/−25% |
| | methane (MSM) | | | |
| Zinc | Zinc Gluconate | 30 | mg | +/−25% |
| | (Zinc-30) | | | |
| Excipients | | 50 | mg | +/−25% |
| Cellulose Gel | | | | |
| Calcium | | | | |
| Stearic Acid | | | | |
| Sodium | | | | |
| Polyethylene | | | | |
| Glycol | | | | |

The method of administration comprises oral delivery of a therapeutic dose of the above-described composition preferably in single tablet form on a daily basis for a person of average size.

A clinical investigation was conducted using a placebo-controlled double-blind ingestion study followed by microscopic three-dimensional imaging analysis in dry skin in 33 subjects who always tended to have rough skin due to dryness. Results show a significant improvement of dryness and itching of the skin, increase in water content, and marked improvement to smoothness, exfoliation, and texture of the skin. The above findings evidence the fact that long-term ingestion of an oral supplement as described is effective in moisture-retention and maintaining smoothness, health and aesthetics of the skin.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

1. A composition for treating skin disorders comprising:
at least one sphingolipid; and
an ingestible sulfur compound.

2. The composition according to claim 1, wherein said at least one sphingolipid comprises a ceramide.

3. The composition according to claim 2, wherein said ceramide comprises a plant-derived phytoceramide.

4. The composition according to claim 3, wherein said ceramide comprises a rice-derived phytoceramide.

5. The composition according to claim 1, wherein said at least one sphingolipid comprises approximately 9.5% parts per weight of at least one sphingolipid.

6. The composition according to claim 1, wherein said sulfur comprises any one from among the group consisting of organosulfur compound, sulfur salt or organosulfur moiety.

7. The composition according to claim 5, wherein said sulfur comprises any one from among the group consisting of cadmium sulfide, zinc pyridinium-1-thiol N-oxide (Zpt), triclosan, halocarban, menthone, undecylene acid, resorcinol, isopropylmethylphenol, salicylic acid, cysteine and methionine.

8. The composition according to claim 1, wherein said sulfur comprises approximately 71.4% parts per weight of sulfur.

9. The composition according to claim 1, further comprising at least one adjuvant.

10. The composition according to claim 1, wherein said at least one adjuvant comprises zinc.

11. The composition according to claim 10, wherein said zinc adjuvant comprises zinc methionine.

12. The composition according to claim 1, further comprising a plurality of excipients.

13. A composition for treating skin disorders comprising:
at least one sphingolipid; and
zinc.

14. The composition according to claim 17, wherein said zinc comprises zinc methionine.

15. A method of administration of the composition of claim 1 comprising a step of orally ingesting said composition once per day.

16. A method of administration of the composition of claim 15 comprising a step of orally ingesting one tablet of said composition once per day.

17. The method according to claim 15, wherein said at least one sphingolipid comprises approximately 9.5% parts per weight of at least one sphingolipid.

18. The method according to claim 15, wherein said sulfur comprises approximately 71.4% parts per weight of pure Methylsulfonylmethane (MSM).

19. A composition for treating acne comprising:
40 mg+/−50% of one or more sphingolipids;
300+/−25% of a sulfur-containing substance.

20. The composition of claim 19 further comprising 30 mg+/−25% zinc gluconate.
